(19) United States
(12) Patent Application Publication
(10) Pub. No.: US 2026/0345934 A1
(43) Pub. Date: Nov. 5, 2026

(54) AUTOMATIC OXYGEN SUPPLY REGULATION SYSTEM

(71) Applicant: Taipei Medical University, Taipei City (TW)

(72) Inventors: Pai-Chien Chou, Taipei City (TW); Chien-Hua Chen, Taipei City (TW); Hsueh-Lien Su, Taipei City (TW); Ya-Ting Juang, Taipei City (TW)

(21) Appl. No.: 19/273,203

(22) Filed: Jul. 18, 2025

Related U.S. Application Data
(60) Provisional application No. 63/673,277, filed on Jul. 19, 2024.

(30) Foreign Application Priority Data
Sep. 9, 2024 (TW) 113134004

(51) Int. Cl. A61M 16/00; A61B 5/00; A61B 5/113; A61B 5/145

(52) U.S. Cl. CPC A61M 16/026; A61B 5/1135; A61B 5/14542; A61B 5/4839; A61M 2202/0208

(57) ABSTRACT

Provided is an automatic oxygen supply regulation system, including an oxygen saturation detection unit, strap assembly, oxygen supply regulating unit, and processing unit. The oxygen saturation detection unit detects the user's oxygen saturation. The strap assembly obtains strap parameter changes caused by the user's breathing. The oxygen supply regulating unit regulates oxygen supply from an oxygen supply source to a user interface unit. The processing unit determines an oxygen optimal flow required based on the oxygen saturation and establishes correlation between tidal volume and strap parameter changes based on the tidal volume and strap parameter changes of the user corresponding to different breathing states. The processing unit further determines an oxygen target flow according to the user's real-time breathing status based on the correlation and controls the oxygen supply regulating unit to regulate the oxygen supply to the user interface unit to achieve the oxygen target flow.

### TECHNICAL FIELD

The present disclosure relates to the technical field of oxygen supply, and in particular, to an automatic oxygen supply regulation system that applies strap parameter measurement and the tidal volume changes it represents, combined with detection of oxygen saturation, to determine oxygen supply flow rate for reducing oxygen consumption.

### BACKGROUND

For many populations suffering from low oxygen saturation symptoms, such as patients with poor cardiopulmonary function, pulmonary fibrosis, pneumonia and chronic obstructive pulmonary disease (COPD), high-intensity exercise groups, and mountain climbing groups, hypoxia may occur before, during, or after activities due to daily life activities, altitude, or exercise processes. Therefore, if oxygen can be appropriately supplied to meet the body's needs, it will be possible to avoid physical damage caused by hypoxia.

Conventional oxygen supply systems are mostly designed to continuously provide oxygen; however, since users only need oxygen to enter the lungs for oxygen exchange during the inspiration phase of each breathing cycle, there is no need for oxygen during the expiration phase and the breath-holding phase. Therefore, for oxygen supply systems that continuously provide oxygen, oxygen is still supplied to users uninterruptedly during the expiration phase and the breath-holding phase, which easily leads to waste of oxygen usage and causes reduction in the available oxygen supply time.

Furthermore, conventional oxygen supply systems mostly provide a fixed oxygen supply amount each time oxygen is supplied. If the user is in an exercise state or other different physiological conditions, the increase in exercise intensity may cause an increase in oxygen consumption, and when an increase in the oxygen supply amount is needed, conventional oxygen supply systems cannot promptly reflect the user's needs, which may cause an insufficient oxygen supply amount, thereby leading to user hypoxia and further causing potential physical damage such as myocardial infarction and lactic acid accumulation.

Therefore, how to design an automatic oxygen supply regulation system that can effectively improve the aforementioned problems is indeed an urgent issue that needs to be resolved at present.

### SUMMARY

The present disclosure provides an automatic oxygen supply regulation system that performs automatic control of oxygen supply flow rate by evaluating the user's tidal volume and thoracic changes in different breathing states.

According to the aforementioned automatic oxygen supply regulation system, the present disclosure enables dynamic advance regulation of the supplied oxygen flow rate based on the user's different tidal volume demands corresponding to different exercise intensities when the user is in a resting breathing state or in an exercise breathing state, so as to maintain stable oxygen saturation of the user.

According to the aforementioned automatic oxygen supply regulation system, the present disclosure applies a strap assembly to measure thoracic changes of the user in different breathing states to determine the inspiration phase and the expiration phase, thereby providing oxygen supply only during the inspiration phase.

In at least one embodiment, the automatic oxygen supply regulation system of the present disclosure comprises an oxygen saturation detection unit, a strap assembly, an oxygen supply regulating unit, and a processing unit. The oxygen saturation detection unit is configured to detect oxygen saturation of a user. The strap assembly is configured to obtain a strap parameter change caused by breathing of the user. The oxygen supply regulating unit is configured to regulate an oxygen supply from an oxygen supply source to a user interface unit. The processing unit is configured to determine an optimal flow of oxygen required based on the oxygen saturation and establish a correlation between a tidal volume change and the strap parameter change based on the strap parameter changes corresponding to different breathing states of the user. The processing unit is configured to further determine an oxygen target flow according to a real-time breathing status of the user based on the correlation and control the oxygen supply regulating unit to regulate the oxygen supply to the user interface unit to achieve the oxygen target flow.

In at least one embodiment of the present disclosure, the processing unit is further configured to establish the correlation between the tidal volume change and the strap parameter change based on the tidal volume change and the strap parameter change of the user in a resting breathing state and in an exercise breathing state.

In at least one embodiment of the present disclosure, the strap parameter change is at least one of a change in length and a change in tension of the strap assembly.

In at least one embodiment of the present disclosure, the oxygen saturation detection unit is configured to determine the optimal flow of oxygen to be supplied by detecting the oxygen saturation of the user in a resting breathing state and to use a corresponding strap parameter change at that time as a baseline reference for tidal volume breathing.

In at least one embodiment of the present disclosure, the processing unit is configured to estimate an oxygen flow increase ratio required by the user in an exercise breathing state based on the correlation between the tidal volume change and the strap parameter change and to determine the oxygen target flow based on the optimal flow of oxygen and the oxygen flow increase ratio.

In at least one embodiment, the automatic oxygen supply regulation system of the present disclosure further comprises a first conduit and a second conduit, wherein the first conduit is in fluid communication at both ends with the oxygen supply regulating unit and the oxygen supply source, respectively, and the second conduit is in fluid communication at both ends with the oxygen supply regulating unit and the user interface unit, respectively.

In at least one embodiment, the automatic oxygen supply regulation system of the present disclosure further comprises a third conduit, wherein the third conduit is in fluid communication at both ends with the oxygen supply regulating unit and ambient air, respectively, and wherein the processing unit is configured to control the oxygen supply regulating unit to introduce the ambient air from the third conduit and mix the ambient air introduced from the third conduit into the second conduit to adjust an oxygen percentage after mixing.

In at least one embodiment of the present disclosure, the processing unit is further configured to determine an inspiration phase and an expiration phase based on the strap parameter changes generated by the strap assembly in response to the breathing, and the processing unit is configured to control the oxygen supply regulating unit to provide the oxygen supply during the inspiration phase and to stop the oxygen supply during the expiration phase.

In at least one embodiment of the present disclosure, the oxygen supply regulating unit comprises a first flow valve, and the processing unit is configured to send a control signal to the oxygen supply regulating unit during the inspiration phase to regulate at least one of an opening duration and an opening degree of the first flow valve and to send a closing signal to the oxygen supply regulating unit during the expiration phase to close the first flow valve.

In at least one embodiment of the present disclosure, the tidal volumes corresponding to different breathing states of the user and the correlation between the tidal volume change and the strap parameter change are obtained after performing a pulmonary function measurement on the user. The pulmonary function measurement can measure the tidal volume of the user. During operation, the user is requested to perform resting breathing or deep breathing to confirm the correlation between the tidal volume change and the strap parameter change; that is, when the tidal volume represented by the measured strap parameter changes can be determined, it is possible to predict the tidal volume under each strap parameter change and correspondingly increase oxygen flow rate to reduce the risk of hypoxia caused by insufficient oxygen supply.

In at least one embodiment of the present disclosure, the strap assembly comprises a first strap, and the first strap is configured to obtain the strap parameter change generated by a chest area of the user in response to the breathing.

In at least one embodiment of the present disclosure, the strap assembly further comprises a second strap, and the second strap is configured to obtain the strap parameter change generated by an abdominal area of the user adjacent to the chest area in response to the breathing.

### BRIEF DESCRIPTION OF THE DRAWINGS

The present disclosure can be more fully understood by reading the following description of specific embodiments and referring to the accompanying drawings.

FIG. 1 is a schematic diagram of the automatic oxygen supply regulation system of the present disclosure.

FIG. 2 is a schematic diagram of another embodiment of the automatic oxygen supply regulation system of the present disclosure.

FIG. 3 is a schematic diagram of yet another embodiment of the automatic oxygen supply regulation system of the present disclosure.

### DETAILED DESCRIPTION

Since the various aspects and embodiments are merely illustrative and not limiting, after reading this specification, those having ordinary skill in the art may also understand other aspects and embodiments without departing from the scope of the present disclosure. According to the following detailed description and claims, the features and advantages of the embodiments will be more clearly apparent.

As used herein, the term “a” or “an” is used to describe the elements and components described herein. This is only for convenience of description and provides a general meaning to the scope of the present disclosure. Therefore, unless clearly indicating otherwise, such description should be understood to include one or at least one, and the singular also includes the plural.

As used herein, the terms “comprising,” “having,” “including,” or any other similar terms are intended to cover non-exclusive inclusion. For example, an element or a structure containing a plurality of elements is not limited to only those elements listed herein and may include other elements not explicitly listed but which are normally inherent to such element or structure.

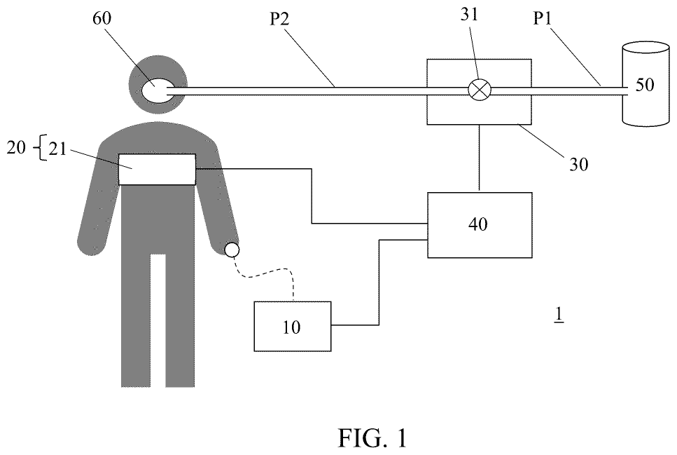

The automatic oxygen supply regulation system of the present disclosure may dynamically regulate oxygen flow supplied from an oxygen supply source to a user interface unit worn by a user based on the user's current breathing state, so as to reduce the user's risk of hypoxia. Please refer to FIG. 1, which is a schematic diagram of the automatic oxygen supply regulation system of the present disclosure. As shown in FIG. 1, the automatic oxygen supply regulation system 1 of the present disclosure comprises an oxygen saturation detection unit 10, a strap assembly 20, an oxygen supply regulating unit 30, and a processing unit 40. In at least one embodiment of the present disclosure, the processing unit 40 is electrically connected to the oxygen saturation detection unit 10, the strap assembly 20, and the oxygen supply regulating unit 30. In at least one embodiment of the present disclosure, the processing unit 40 may perform bidirectional signal transmission with the oxygen saturation detection unit 10, the strap assembly 20, and the oxygen supply regulating unit 30, respectively, through wireless or wired transmission methods.

In at least one embodiment of the present disclosure, the oxygen saturation detection unit 10 may detect the user's oxygen saturation in real-time or periodically. In some embodiments of the present disclosure, the oxygen saturation detection unit 10 may employ a common oxygen saturation monitor or a similar device, which is not particularly limited in the present disclosure. In some embodiments of the present disclosure, the oxygen saturation detection unit 10 may detect the user's oxygen saturation under different breathing states (such as a resting breathing state or an exercise breathing state) and assist the system as a criterion for determining whether a basic oxygen supply should be additionally increased. The aforementioned resting breathing state can be defined as breathing actions performed by the user under normal resting conditions, while the exercise breathing state can be defined as breathing actions performed by the user during exercise or activity. For example, by obtaining the user's oxygen saturation under different breathing states and cross-comparing with the oxygen flow rate supplied in corresponding states, the optimal flow of oxygen required to be supplied to the user in corresponding states can be determined to meet the oxygen saturation requirements under such state.

In at least one embodiment of the present disclosure, the strap assembly 20 may obtain strap parameter changes caused by the user's breathing. In some embodiments of the present disclosure, the strap assembly 20 comprises a first strap 21, and the first strap 21 is configured to be partially or completely wrapped around and fixed to the user's chest area to obtain strap parameter changes generated by the user's chest area in response to breathing. In some embodiments of the present disclosure, the body of the first strap 21 may be made of a material having elastic stretchability, and one or more sensing elements may be disposed inside the first strap 21. In some embodiments of the present disclosure, the first strap 21 undergoes elastic deformation with the rise and fall of the user's chest area caused by breathing, thereby generating strap parameter changes of the first strap 21. In some embodiments of the present disclosure, the aforementioned strap parameter changes of the first strap 21 may be a change in length and/or a change in tension of the first strap 21. Therefore, the strap parameter changes detected by the aforementioned sensing elements can serve as a reference basis for subsequent oxygen supply.

For example, in some embodiments of the present disclosure, the aforementioned strap parameter changes may serve as a basis for subsequently determining whether the user is in an inspiration phase or in an expiration phase of breathing. In some embodiments of the present disclosure, the thoracic changes reflected by the aforementioned strap parameter changes may serve as a reference for subsequently evaluating tidal volume changes when the user breathes. In some embodiments of the present disclosure, thoracic changes may be obtained by measuring and calculating changes in the user's thoracic circumference, chest diameter, chest major axis, chest minor axis, or any combination thereof, and the present disclosure is not limited thereto.

In at least one embodiment of the present disclosure, the oxygen supply regulating unit 30 is in fluid communication with an oxygen supply source 50 and a user interface unit 60 worn by the user, respectively, to regulate an oxygen supply from the oxygen supply source 50 to the user interface unit 60. In some embodiments of the present disclosure, the oxygen supply regulating unit 30 may comprise a first flow valve 31, which is configured to control the communication state between a gas conduit connected to the oxygen supply source 50 and a gas conduit connected to the user interface unit 60. In some embodiments of the present disclosure, the oxygen supply regulating unit 30 may receive signals from the processing unit 40 to control the opening and closing of the first flow valve 31 and/or to regulate the opening degree and/or opening duration of the first flow valve 31. In some embodiments, the automatic oxygen supply regulation system 1 of the present disclosure further comprises a first conduit P1 and a second conduit P2, wherein the first conduit P1 is in fluid communication at both ends with the first flow valve 31 of the oxygen supply regulating unit 30 and the oxygen supply source 50, respectively, and the second conduit P2 is in fluid communication at both ends with the first flow valve 31 of the oxygen supply regulating unit 30 and the user interface unit 60, respectively, such that the oxygen supply regulating unit 30 may receive oxygen from the oxygen supply source 50 via the first conduit P1 and deliver oxygen toward the user interface unit 60 via the second conduit P2.

In at least one embodiment of the present disclosure, the oxygen supply source 50 may supply oxygen, and the oxygen supply source 50 may be a general oxygen cylinder, a portable oxygen tank, or an oxygen supply system of a medical facility, which is not particularly limited in the present disclosure. In at least one embodiment of the present disclosure, the user interface unit 60 may be worn by the user and to output oxygen from the oxygen supply source 50, and the user interface unit 60 may be a nasal cannula or a breathing mask, which is not particularly limited in the present disclosure.

In at least one embodiment of the present disclosure, the processing unit 40 may receive the oxygen saturation measured by the oxygen saturation detection unit 10 to determine the optimal flow of oxygen required to be supplied to the user based on the oxygen saturation. For example, in some embodiments of the present disclosure, the processing unit 40 may detect the user's oxygen saturation in a resting breathing state through the oxygen saturation detection unit 10 and cross-compare with the oxygen flow rate supplied in the resting breathing state to determine the optimal flow of oxygen required to be supplied to the user to meet the oxygen saturation requirements in the resting breathing state. In some embodiments of the present disclosure, the processing unit 40 may be configured as hardware (such as a computer host, a server, a processor, or a similar device), software (such as computer software or application programs), or a combination of the aforementioned hardware and software.

In at least one embodiment of the present disclosure, the processing unit 40 may establish a correlation between the tidal volume change and the strap parameter change based on the strap parameter changes corresponding to different breathing states of the user. In some embodiments of the present disclosure, when the user first uses the automatic oxygen supply regulation system 1 of the present disclosure, a pulmonary function measurement may first be performed in coordination to obtain the user's tidal volume and strap parameter changes in a resting breathing state and to obtain the maximum amount of thoracic circumference expansion through deep breathing as the user's tidal volume and strap parameter changes in an exercise breathing state. Accordingly, the processing unit 40 may apply relevant formulas to estimate the correlation between the tidal volume change and the strap parameter change. For example, in some embodiments of the present disclosure, based on the thoracic circumference changes generated by the user's breathing, proportional relationships with the strap parameter changes and the thoracic volume changes may be established, respectively, thereby estimating the correlation between the tidal volume change and the strap parameter change.

Specifically, in some embodiments of the present disclosure, since the user's thoracic changes may represent changes in thoracic circumference, if the thorax is ideally estimated to be circular, the calculation method for thoracic circumference can be: 2πr, where r is the radius of the circle. In addition, the user's breathing represents volume changes, which can theoretically be calculated as: πr²×thoracic height. Since the thoracic height of the same user is generally fixed, based on the aforementioned calculation formula combined with the measured change ratio of thoracic circumference, the quadratic concept can be further utilized to calculate tidal volume changes. For example, assuming the thoracic circumference change is 1.2 times, the tidal volume change is approximately 1.44 times the original tidal volume; and assuming the thoracic circumference change is 1.5 times, the tidal volume change is approximately 2.25 times the original tidal volume. The aforementioned existing calculation formulas can be applied to most users; however, since the user's related thoracic volume is affected by organs within the thoracic cavity, the final conversion ratio will still be calibrated through the aforementioned pulmonary function measurement. In addition, the correlation between the thoracic volume change and the tidal volume change can also refer to published journal papers (such as Padkao et al., “Relationships between respiratory muscle strength, chest wall expansion, and functional capacity in healthy nonsmokers,” Journal of Exercise Rehabilitation 2020; 16(2): 189-196).

In clinical applications, users may perform a pulmonary function measurement through standard pulmonary function measurement instruments, and during the measurement process, users are required to perform shallow breathing or deep breathing to respectively represent the lung volume required by the user in a resting breathing state and in an exercise breathing state. The aforementioned measured values can correspond to volume change prediction values under thoracic changes, and through the processing unit 40 applying Boyle's law for related calculations, the proportional relationship between the tidal volume change and the strap parameter change (i.e., the aforementioned correlation) can be obtained, thereby serving as a relevant conversion basis for the oxygen flow rate required to be supplied subsequently.

In at least one embodiment of the present disclosure, the processing unit 40 may determine an oxygen target flow according to the user's real-time breathing status based on the aforementioned correlation between the tidal volume change and the strap parameter change and control the oxygen supply regulating unit 30 to regulate the oxygen supply to achieve the oxygen target flow. In some embodiments of the present disclosure, the processing unit 40 may estimate an oxygen flow increase ratio required by the user in a real-time breathing status based on the correlation between the tidal volume change and the strap parameter change to determine the oxygen target flow based on the optimal flow of oxygen and the oxygen flow increase ratio. For example, when the user suddenly enters an exercise breathing state and the oxygen demand increases significantly, the automatic oxygen supply regulation system 1 of the present disclosure may estimate the oxygen flow increase ratio corresponding to the user's real-time breathing status based on the aforementioned correlation, use the set optimal flow of oxygen as the basic supply flow rate, calculate the oxygen flow rate that should be increased in coordination with the oxygen flow increase ratio, and determine the final oxygen target flow by adding the two together. Accordingly, the processing unit 40 may send a signal to the oxygen supply regulating unit 30 to control the oxygen supply regulating unit 30 to regulate the current oxygen supply to the oxygen target flow.

As mentioned above, in some embodiments, the automatic oxygen supply regulation system 1 of the present disclosure may measure and record strap parameter changes during a resting breathing state, strap parameter changes during inspiration and/or expiration, and strap parameter changes during an exercise breathing state for the user through the strap assembly 20. Accordingly, the processing unit 40 may correspondingly evaluate changes in the user's thoracic circumference through strap parameter changes measured under different states, thereby determining the inspiration phase and the expiration phase of the user's breathing. In some embodiments of the present disclosure, the processing unit 40 may control the oxygen supply regulating unit 30 to provide an oxygen supply only during the inspiration phase, while stopping an oxygen supply during the expiration phase, so as to save oxygen consumption and extend oxygen supply time. Specifically, in some embodiments of the present disclosure, the processing unit 40 may send a control signal to the oxygen supply regulating unit 30 during the inspiration phase to regulate the opening duration and/or opening degree of the first flow valve 31, thereby achieving the required oxygen target flow to be supplied; in other embodiments, the processing unit 40 may send a closing signal to the oxygen supply regulating unit 30 during the expiration phase to reduce or even close the first flow valve 31, thereby achieving the effect of saving excessive oxygen consumption.

For example, the oxygen supply flow rate calculated by the processing unit 40 may use a fixed oxygen fraction of normal oxygen supply as an estimation target. Since the proportion of pure oxygen in air is 21%, the proportion of oxygen entering the lungs will affect the final oxygen saturation while the supplied oxygen is pure oxygen. Specifically, assuming the user uses 2 liters per minute of pure oxygen supply (i.e., 2 L/min), the pure oxygen supply per second at this time is 33.33 mL (i.e., 33.33 mL/sec); further assuming the user's tidal volume is 300 mL, after deducting 33.33 mL of pure oxygen, the remaining mixed air volume is 266.67 mL, and the proportion of oxygen entering the lungs during each breath after conversion will be: (33.33×100%+266.67×21%)/300=29.7769%, which is the so-called fraction of inspired oxygen (abbreviated as FiO₂).

To enable users to maintain sufficient oxygen saturation in an exercise breathing state, in response to the increased air volume (FiO₂ of 21%), the corresponding oxygen supply should be increased to maintain FiO₂ at 29.7769%. Assuming the user's tidal volume increases to 400 mL at this time, to maintain the aforementioned required FiO₂, the pure oxygen supply per second will be: 400 mL×29.7769%=119.1076 mL. For conventional oxygen supply systems, the pure oxygen supply per minute at this time will be: 119.1076 mL/sec×60 sec=7146.456 mL, which is approximately 7.146456 L of pure oxygen provided per minute. In contrast, the automatic oxygen supply regulation system 1 of the present disclosure supplies oxygen only during the inspiration phase. When assuming the user's breathing rate increases to 25 breaths per minute during exercise and the same pure oxygen supply is maintained at 119.107 mL/sec, with each inspiration lasting approximately 1 second and a total inspiration time of approximately 25 seconds per minute, only approximately 2977.675 mL of oxygen supply need to be consumed per minute, equivalent to approximately 2.977675 L. Accordingly, the automatic oxygen supply regulation system 1 of the present disclosure can maintain the corresponding oxygen supply requirements. Also, since oxygen is provided only during the inspiration phase, the oxygen consumption per minute can be reduced from 7.146456 liters to 2.977675 liters, requiring only approximately 40% of oxygen consumption, significantly reducing oxygen loss by approximately 60%, thereby greatly improving the usable time of oxygen.

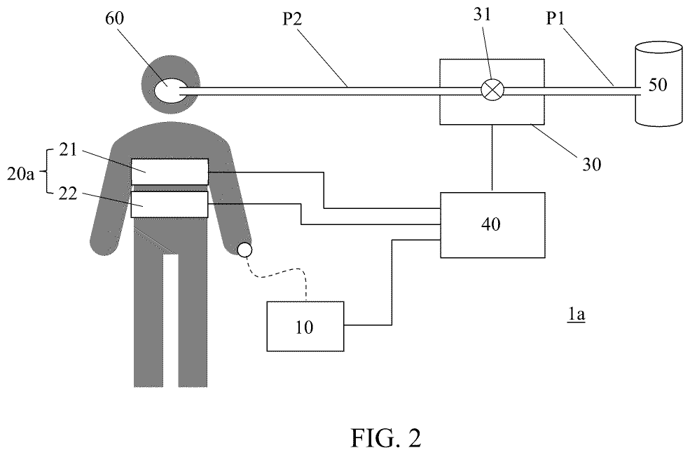

Please further refer to FIG. 2, which is a schematic diagram of another embodiment of the automatic oxygen supply regulation system 1a of the present disclosure. As shown in FIG. 2, in at least one embodiment, the strap assembly 20a of the automatic oxygen supply regulation system 1a of the present disclosure further comprises a second strap 22, and the second strap 22 is configured to be partially or completely wrapped around and fixed to the user's abdominal area adjacent to the chest area (i.e., a position adjacent to the diaphragm) to obtain strap parameter changes generated by the user's corresponding area (such as the rise and fall of the diaphragm) in response to breathing. In some embodiments of the present disclosure, the body of the second strap 22 may also be made of a material having elastic stretchability, and one or more sensing elements are disposed inside the second strap 22. In some embodiments of the present disclosure, the second strap 22 may undergo elastic deformation with the rise and fall of the user's abdominal area caused by breathing, thereby generating strap parameter changes of the second strap 22. In some embodiments of the present disclosure, the aforementioned strap parameter changes of the second strap 22 may be a change in length and/or a change in tension of the second strap 22.

Many users habitually use abdominal breathing during respiration, causing the user to experience both chest area fluctuations and abdominal area fluctuations during breathing, particularly in an exercise breathing state. Since the chest area fluctuations sensed by the first strap 21 will vary with the user's different body types and/or different chest fixation positions of the first strap 21 (e.g., upper chest, middle chest, or lower chest), the strap parameter changes obtained by the second strap 22 that senses the abdominal area fluctuations adjacent to the user's chest area can be combined with the strap parameter changes obtained by the first strap 21 to further calculate related volume changes and tidal volume changes, thereby improving the accuracy of estimating the correlation between the aforementioned tidal volume changes and strap parameter changes.

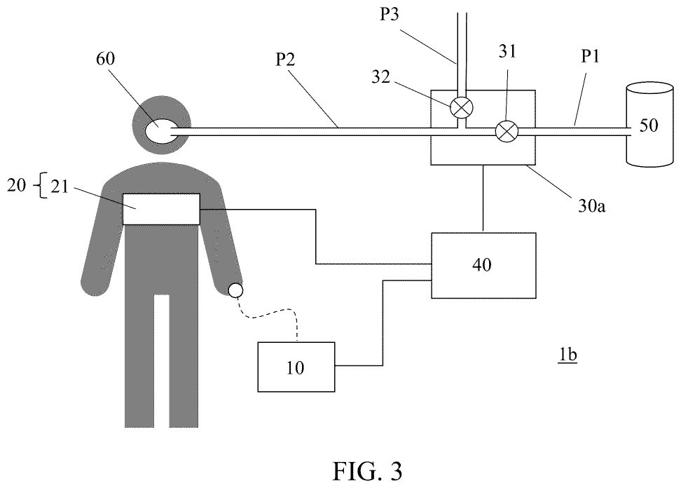

Please further refer to FIG. 3, which is a schematic diagram of yet another embodiment of the automatic oxygen supply regulation system 1b of the present disclosure. As shown in FIG. 3, in at least one embodiment, the automatic oxygen supply regulation system 1b of the present disclosure further comprises a third conduit P3. In at least one embodiment of the present disclosure, the third conduit P3 is in fluid communication at both ends with the oxygen supply regulating unit 30a and ambient air, respectively. Accordingly, the processing unit 40 may not only control the oxygen supply regulating unit 30a to input oxygen from the oxygen supply source 50 via the first conduit P1, but also control the oxygen supply regulating unit 30a to input ambient air from the ambient environment via the third conduit P3 and mix the ambient air input from the ambient environment into the second conduit P2, followed by delivering a mixed gas of oxygen and ambient air toward the user interface unit 60 via the second conduit P2. In some embodiments of the present disclosure, the oxygen supply regulating unit 30a further comprises a second flow valve 32, which is configured to control the communication state between the third conduit P3 connected to the ambient environment and the second conduit P2 connected to the user interface unit 60. In some embodiments of the present disclosure, the oxygen supply regulating unit 30a may receive signals from the processing unit 40 to control the opening and closing of the second flow valve 32 and/or to regulate the opening duration and/or opening degree of the second flow valve 32.

As mentioned above, in some embodiments of the present disclosure, the processing unit 40 may send signals to control the oxygen supply regulating unit 30a to regulate the opening duration and/or opening degree of the first flow valve 31 and the second flow valve 32, thereby controlling the oxygen flow rate supplied from the oxygen supply source 50 to the user interface unit 60 and/or the ambient air flow rate mixed into the oxygen, so as to regulate the oxygen proportion after oxygen and ambient air are mixed.

In some embodiments of the present disclosure, when using any of the aforementioned automatic oxygen supply regulation systems 1, 1a, or 1b of the present disclosure, the user may first wear the strap assembly 20 or 20a and utilize the oxygen saturation detection unit 10 to detect the user's oxygen saturation to determine the optimal flow of oxygen required to be supplied; then, the user may coordinate with a pulmonary function measurement to obtain tidal volume changes and strap parameter changes under different breathing states; afterward, the processing unit 40 may estimate the proportional relationship between the tidal volume changes and the strap parameter changes, apply Boyle's law, determine the oxygen target flow required for the user's current breathing state, and finally control the oxygen supply regulating unit 30 or 30a to regulate the oxygen supply from the currently supplied oxygen flow rate to the oxygen target flow. Since the oxygen flow rate provided by the automatic oxygen supply regulation system of the present disclosure is dynamically controlled and may preemptively increase the oxygen supply value in response to the user's different physiological conditions, it can avoid insufficient oxygen supply under a high oxygen demand, thereby preventing cardiopulmonary problems that may be caused by the user's hypoxia risk.

Through the automatic oxygen supply regulation system of the present disclosure, users may monitor whether the user's oxygen saturation is normal through the oxygen saturation detection unit and use the strap assembly for a proportion assessment of inspiration and expiration during breathing to preemptively increase the oxygen supply flow rate under conditions of increased tidal volume when the user exercises or is active; if oxygen saturation still decreases during user's exercise or activity on the premise that the aforementioned oxygen supply flow rate has been increased, the system will additionally increase the oxygen supply flow rate. In addition, the automatic oxygen supply regulation system of the present disclosure may also predict the inspiration phase and the expiration phase through the strap assembly and provide oxygen supply only during the inspiration phase, thereby effectively reducing the inappropriate consumption of oxygen that continues to be supplied during the expiration phase in conventional technology. This extends the supply time of fixed-amount oxygen supply such as oxygen tanks, while avoiding user harm caused by excessive or insufficient oxygen supply. The automatic oxygen supply regulation system of the present disclosure may also apply the enlarged dimensions of thoracic circumference and/or diameter during inspiration, combined with a pulmonary function measurement, to determine the correspondence between the tidal volume changes and the strap parameter changes during inspiration under different states, thereby deciding whether to increase oxygen output during oxygen supply. For example, during user's exercise, the oxygen supply flow rate may be automatically increased in real-time to reduce the risk of hypoxia.

Accordingly, the automatic oxygen supply regulation system of the present disclosure may provide oxygen only when determining that the user is in the inspiration phase, thereby effectively reducing oxygen consumption. Furthermore, the automatic oxygen supply regulation system of the present disclosure may calculate the appropriate oxygen supply ratio that should be increased for the user when needed by detecting thoracic differences caused by changes in the user's breathing depth through the strap assembly, thereby preemptively increasing oxygen supply to reduce hypoxia risk. The automatic oxygen supply regulation system of the present disclosure may be applied to disease populations, mountain climbing populations, exercise populations, and other groups requiring oxygen supply. Furthermore, through detection of respiratory movement by the strap assembly, it may provide information on whether insufficient oxygen saturation under oxygen supply is due to insufficient inspiration intensity and may provide clinical warnings to inform peripheral or backend users of abnormal oxygen supply conditions. In addition, the automatic oxygen supply regulation system of the present disclosure may reduce clinical risks, improve user's performance during exercise, and reduce adverse effects that may be caused to the cardiopulmonary system during exercise. Meanwhile, the automatic oxygen supply regulation system of the present disclosure may also be used in medical facilities to improve the safety of oxygen supply for hospitalized patients, avoid harm caused by excessive or insufficient oxygen supply, and prevent potential problems of sudden patient death.

The above embodiments are merely illustrative in nature and are not intended to limit the embodiments of the claimed subject matter or the applications of such embodiments. Furthermore, although at least one exemplary embodiment has been presented in the foregoing embodiments, it should be understood that numerous variations may still exist in the present disclosure. It should also be understood that the embodiments described herein are not intended to limit the scope, use, or configuration of the claimed subject matter in any way. Rather, the foregoing embodiments will provide those having ordinary skill in the art with a convenient guide for implementing one or more of the described embodiments. Moreover, various changes may be made in the function and arrangement of elements without departing from the scope defined by the claims, and the claims include known equivalents and all foreseeable equivalents at the time of filing this patent application.

What is claimed is:

1. An automatic oxygen supply regulation system for regulating oxygen flow supplied from an oxygen supply source to a user interface unit worn by a user, the automatic oxygen supply regulation system comprising:
an oxygen saturation detection unit configured to detect oxygen saturation of the user;
a strap assembly configured to obtain a strap parameter change caused by breathing of the user;
an oxygen supply regulating unit configured to be in fluid communication with the oxygen supply source and the user interface unit, respectively, and configured to regulate an oxygen supply from the oxygen supply source to the user interface unit; and
a processing unit electrically connected to the oxygen saturation detection unit, the strap assembly, and the oxygen supply regulating unit, the processing unit being configured to determine an optimal flow of oxygen required based on the oxygen saturation and to establish a correlation between a tidal volume change and the strap parameter change based on the strap parameter changes corresponding to different breathing states of the user, and the processing unit being further configured to determine an oxygen target flow according to a real-time breathing status of the user based on the correlation and to control the oxygen supply regulating unit to regulate the oxygen supply to the user interface unit to achieve the oxygen target flow.

2. The automatic oxygen supply regulation system of claim 1, wherein the processing unit is further configured to establish the correlation between the tidal volume change and the strap parameter change based on the tidal volume change and the strap parameter change of the user in a resting breathing state and in an exercise breathing state.

3. The automatic oxygen supply regulation system of claim 1, wherein the strap parameter change is at least one of a change in length and a change in tension of the strap assembly.

4. The automatic oxygen supply regulation system of claim 1, wherein the oxygen saturation detection unit is configured to determine the optimal flow of oxygen to be supplied by detecting the oxygen saturation of the user in a resting breathing state and to use a corresponding strap parameter change at that time as a baseline reference for tidal volume breathing.

5. The automatic oxygen supply regulation system of claim 4, wherein the processing unit is further configured to estimate an oxygen flow increase ratio required by the user in an exercise breathing state based on the correlation between the tidal volume change and the strap parameter change and to determine the oxygen target flow based on the optimal flow of oxygen and the oxygen flow increase ratio.

6. The automatic oxygen supply regulation system of claim 1, further comprising a first conduit and a second conduit, wherein the first conduit is in fluid communication at both ends with the oxygen supply regulating unit and the oxygen supply source, respectively, and the second conduit is in fluid communication at both ends with the oxygen supply regulating unit and the user interface unit, respectively.

7. The automatic oxygen supply regulation system of claim 6, further comprising a third conduit, wherein the third conduit is in fluid communication at both ends with the oxygen supply regulating unit and ambient air, respectively, and wherein the processing unit is configured to control the oxygen supply regulating unit to introduce the ambient air from the third conduit and mix the ambient air introduced from the third conduit into the second conduit to adjust an oxygen percentage after mixing.

8. The automatic oxygen supply regulation system of claim 1, wherein the processing unit is further configured to determine an inspiration phase and an expiration phase based on the strap parameter change generated by the strap assembly in response to the breathing, and wherein the processing unit is configured to control the oxygen supply regulating unit to provide the oxygen supply during the inspiration phase and to stop the oxygen supply during the expiration phase.

9. The automatic oxygen supply regulation system of claim 8, wherein the oxygen supply regulating unit comprises a first flow valve, and wherein the processing unit is configured to send a control signal to the oxygen supply regulating unit during the inspiration phase to regulate at least one of an opening duration and an opening degree of the first flow valve and to send a closing signal to the oxygen supply regulating unit during the expiration phase to close the first flow valve.

10. The automatic oxygen supply regulation system of claim 1, wherein tidal volumes corresponding to different breathing states of the user and the correlation between the tidal volume change and the strap parameter change are obtained after performing a pulmonary function measurement on the user.

11. The automatic oxygen supply regulation system of claim 1, wherein the strap assembly comprises a first strap, and the first strap is configured to obtain the strap parameter change generated by a chest area of the user in response to the breathing.

12. The automatic oxygen supply regulation system of claim 11, wherein the strap assembly further comprises a second strap, and the second strap is configured to obtain the strap parameter change generated by an abdominal area of the user adjacent to the chest area in response to the breathing.
